(19) United States
(12) Patent Application Publication
(10) Pub. No.: US 2022/0067864 A1
(43) Pub. Date: Mar. 3, 2022

(54) DE-IDENTIFIED MEDICAL RECORDS AND RELATED SYSTEMS

(71) Applicant: Envision Diagnostics, Inc., El Segundo, CA (US)

(72) Inventors: Alexander C. Walsh, Los Angeles, CA (US); Richard Castro, Santa Monica, CA (US); Paul G. Updike, Cerritos, CA (US); Adam Andrew Doherty, Fallbrook, CA (US); Johan Prinsloo, Redondo Beach, CA (US)

(21) Appl. No.: 16/855,862

(22) Filed: Apr. 22, 2020

Related U.S. Application Data
(60) Provisional application No. 62/837,663, filed on Apr. 23, 2019.

(51) Int. Cl. G06Q 50/26; G06F 16/23; G16H 10/60; G06F 21/62; A61B 3/12; A61B 3/00

(52) U.S. Cl. CPC G06Q 50/265; G06F 16/2379; G16H 10/60; G16H 40/67; A61B 3/12; A61B 3/0025; G06F 21/6245

(57) ABSTRACT

An electronic healthcare data de-identification system can accept data containing personally-identifiable information and output de-identified healthcare data that is uniquely identifiable and has preserved temporal order. The unique identity of the de-identified healthcare data is preserved with universally unique identifiers and the temporal order of the de-identified healthcare data is maintained with universally unique sequence values that synchronize the timing of healthcare events. The embodiments described herein make healthcare data more accessible to users without endangering their privacy and allow conclusions to be made from large sets of data without restricting its use due to privacy concerns. Other embodiments are also described.

### INCORPORATION BY REFERENCE TO ANY PRIORITY APPLICATIONS

This application claims the priority benefit of U.S. Application No. 62/837,663, filed Apr. 23, 2019, which is hereby incorporated by reference in its entirety herein.

Any and all applications for which a foreign or domestic priority claim is identified in the Application Data Sheet as filed with the present application are hereby incorporated by reference under 37 C.F.R. § 1.57.

### BACKGROUND

### Field

The present disclosure relates to the field of healthcare, including for example, devices, systems, electronic medical records, data storage, data security, data de-identification, and data analysis. In some but not all instances, these healthcare services may apply only to outpatient encounters, exams, and services.

### Description of the Related Art

Many healthcare providers use electronic medical records to manage patient care. Security is of paramount concern with electronic health records which may contain sensitive, personal information about individuals. However, this same security may introduce barriers to data access for patients and data analysis for providers. Accordingly, systems and methods for improving access to useful healthcare data without endangering the security of private information are presented.

### SUMMARY

Various embodiments of the present disclosure relate to the de-identification of individually-identified healthcare data without losing the unique identity or temporal relationships of the original data. Other uses of the de-identified data are disclosed. Various designs of ophthalmic diagnostic instrumentation are also discussed.

In various embodiments, a system configured to de-identify healthcare data from an ophthalmic diagnostic device comprises a computing system in electronic communication with the ophthalmic diagnostic device, the computing system comprising a processor and computer-executable instructions, wherein the computer-executable instructions, when executed, cause the computing system to accept individually-identifiable eye examination data from the ophthalmic diagnostic device as an input; request and receive at least one ordered value from a remote location in electronic communication with said computing system; the at least one ordered value being based at least in part on the order generated and not based on the specific date or time of the request or the individually-identifiable eye examination data, the at least one ordered value being larger than previously generated ordered values from the remote location, and the at least one ordered value being smaller than future generated ordered values from the remote location; replace at least one date or portion of a date in the individually-identifiable eye examination data with the at least one ordered value; and output de-identified healthcare data incorporating the at least one ordered value.

In various embodiments, a system configured to de-identify healthcare data from an ophthalmic diagnostic device comprises a computing system in electronic communication with the ophthalmic diagnostic device, the computing system comprising a processor and computer-executable instructions, wherein the computer-executable instructions, when executed, cause the computing system to accept individually-identifiable eye examination data from the ophthalmic diagnostic device as an input; request and receive at least one ordered value from a remote location in electronic communication with said computing system; the at least one ordered value being based at least in part on the order generated and not based on the specific date or time of the request or the individually-identifiable eye examination data, the at least one ordered value being larger than ordered values generated from the remote location since the beginning of the calendar year, and the at least one ordered value being smaller than ordered values generated from the remote location for the remainder of the calendar year; replace at least one date or portion of a date in the individually-identifiable eye examination data with the at least one ordered value; and output de-identified healthcare data incorporating the at least one ordered value.

In various embodiments, a system configured to assist in the de-identification of healthcare data comprises a computing system comprising a processor and computer-executable instructions, wherein the computer-executable instructions, when executed, cause the computing system to receive a request across from a remote location to generate at least one ordered value; generate the at least one ordered value such that the value is larger than any previous value generated by said system, is smaller than any future value to be generated by said system and the value is unrelated to the time of the request or calculation; output said ordered value to the remote location in less than one second from receiving the request.

### BRIEF DESCRIPTION OF THE DRAWINGS

The foregoing and other features, aspects and advantages of the present disclosure are described in detail below with reference to the drawings of various embodiments, which are intended to illustrate and not to limit the claimed invention. The drawings comprise the following figures in which:

FIG. 1 is a flowchart showing the process of de-identifying a PHI dataset 100 into a de-identified health information dataset 170.

FIG. 2 is an exemplary table of PHI dataset 100 including unique codes that uniquely but not personally identify this record after de-identification.

FIG. 3 is an example of de-identification table 120 showing one method of encoding de-identification instructions for de-identification system 110. In this table, data fields from a personally-identifiable healthcare dataset are marked for deletion (null), replacement with a unique code or inclusion in a subsequent de-identified health record and the importance or priority of these actions are designated.

FIG. 4 is a table of exemplary records for existing UUID database 130 which acts as a look-up table for UUIDs associated with specific PHI field values. Existing UUID database 130 accumulates data over time as de-identifying system 110 requests new UUIDs for newly-encountered personally-identifiable field values. Each time that de-identifying system 110 requests a new UUID associated with a particular PHI field value, the PHI field value and associated unique code are added to existing UUID database 130 so that de-identifying system 110 can search for and find the same UUID for that PHI field value in the future.

FIG. 5 is a table of exemplary records from new UUID buffer database 140. This database acts as a buffered intermediary between de-identifying system 110 and UUID service 150 to store newly-requested UUIDs or blocks of UUIDs until they are needed by de-identifying system 110. This buffer allows de-identifying system 110 to continue operating normally even when communication between de-identifying system 110 and UUID service 150 is interrupted.

FIG. 6 is an exemplary table of de-identified healthcare dataset 170 produced from the PHI dataset 100 shown in FIG. 2 by de-identifying system 110.

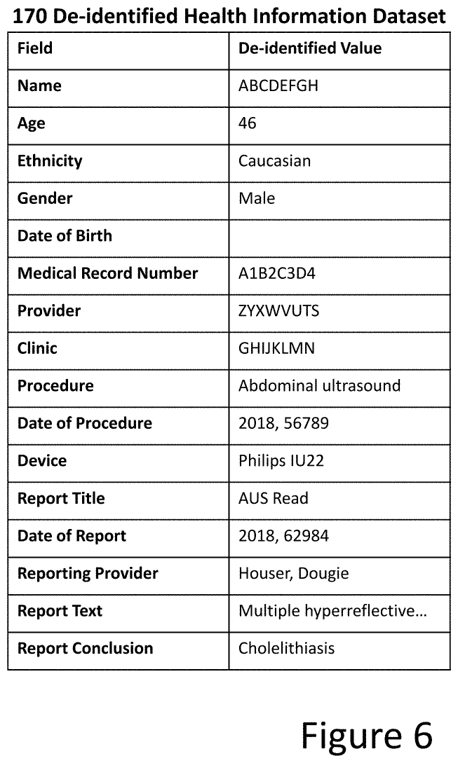

FIG. 7 is a schematic of a process to calculate normal reference intervals from a set of values that is comprised of a mix of normal and abnormal values.

FIG. 8 is a schematic of an automated eye examination device.

### DETAILED DESCRIPTION

Embodiments of the present disclosure will now be described with reference to the accompanying figures, wherein like numerals refer to like elements throughout. The terminology used in the description presented herein is not intended to be interpreted in any limited or restrictive manner, simply because it is being utilized in conjunction with a detailed description of certain specific embodiments of the present disclosure. Furthermore, embodiments of the present disclosure may comprise several novel features, no single one of which is solely responsible for its desirable attributes or which is essential to practicing the embodiments herein described. The embodiments described herein make healthcare data more accessible to users without endangering their privacy and allow conclusions to be made from large sets of data without restricting its use due to privacy concerns.

Medical providers often link health information to the identity of a specific individual during the provision of medical care. This linkage between data, such as examination findings and test results, and an individual's identity is typically necessary in the diagnosis and management of that person's medical condition. A link between health information and an individual's identify may also be helpful in developing and maintaining an historic record of health assessments over time so that providers can, among many other things, monitor changes in health status, analyze the effects of treatments, study disease progression or demonstrate that certain actions were taken at specific times during the management of a patient's health. The link between data and individual identity may also be helpful when studying general associations between health data and patient-specific parameters such as age, gender and ethnicity.

Protected Health Information or PHI can be defined as individually identifiable health information, including demographic data, medical histories, test and laboratory results, mental health conditions, insurance information and other data that a healthcare professional collects to identify and care for an individual. An organized collection of PHI is referred to herein interchangeably as either a PHI database, PHI data or a PHI dataset. The linking of health data to an individual is typically done with patient-specific identifiers, also known as PHI identifiers, individual identifiers or personal identifiers, such as but not limited to: names, geographic subdivisions smaller than a state (except for the initial three digits of a zip code), any part of a date except the year, telephone numbers, vehicle identifiers and serial numbers, license plate numbers, fax numbers, device identifiers and serial numbers, email addresses, web universal resource locators (URLs), social security numbers, internet protocol addresses, medical record numbers, biometric identifiers such as finger and voice prints, health plan beneficiary numbers, full-face photographs and any comparable images, account numbers, certificate or license numbers, or any other unique identifying number, characteristic or code with some exception. Biometric identifiers may be further expanded to include other unique biological measurements such as facial profiles and images of eye structures such as the Palisades of Vogt, the iris or the ocular fundus.

The sensitive nature of the PHI data contained in medical records has resulted in the development of numerous regulations, such as the Health Insurance Portability and Accountability Act (HIPAA), developed in an attempt to protect individuals from inadvertent disclosure of their PHI. It is well recognized that inappropriate disclosure of sensitive information, such as PHI-containing digital health data, has the potential to cause negative consequences for the individuals identified through those data. Therefore, protection of sensitive information is deemed to be important whenever individually-identifying information is associated with it.

In some circumstances, it may be possible or even advantageous to collect sensitive data, such as medical or healthcare data, in a database without associating it with individually-identifying information that could specifically identify the individuals who are the subjects of that data. For example, if the complete and perpetual security of a database cannot be guaranteed, then the potential risk of divulging or disclosing individually identifiable health information through compromise of the database can be mitigated by removing the individually identifiable health information from the database. In other instances, there may be legal or regulatory restrictions on the use and/or transfer of data associated with PHI; and these restrictions may be lessened by the removal of PHI identifiers. In some cases, it may not be legal or ethical to perform research on datasets that contain individually identifiable health information whereas it may be possible to perform the same research on the data if the identities of the research subjects are removed and cannot be discovered or reverse engineered. In addition, in an era when identity theft and public disclosures of personal information are often in the news, patients may feel more secure if they know that their sensitive healthcare data is contained in a dataset that cannot be traced back to them even in the event that the security of the dataset is compromised.

A health information dataset without PHI identifiers may be referred to as a DIHI dataset, DIHI database or simply DIHI data since it contains only de-identified health information (DIHI). In some embodiments, DIHI data may never have contained any PHI identifiers. In embodiments related to the current disclosure, DIHI data is created by the removal of PHI identifiers from a PHI dataset or the replacement of PHI identifiers in a PHI dataset with identifiers that are unique but not personally related to an individual. A DIHI database can be configured as a publicly accessible resource without security or authentication requirements since it does not contain any PHI. A DIHI database may also be configured as a private database with access restricted only to authorized individuals. An access-restricted DIHI database may be further protected by security measures that employ current best practices and are just as strong and effective as the security measures used to secure a database containing PHI.

De-identified health information (DIHI) can be created from personal health information (PHI) by removing, scrubbing, nullifying, replacing or deleting PHI identifiers from the dataset. In addition to having all PHI identifiers removed, the data in a de-identified health information (DIHI) database qualifies as de-identified only if it is generally not useful, alone or in combination with other information, to re-identify the individual. An exception to this last criterion is the provision that a unique code may be associated with DIHI to permit subsequent re-identification provided that the unique code is not derived from or related to information about the individual and cannot otherwise be capable of being translated so as to identify the individual. Stated another way, for a unique code to qualify as a part of DIHI, it must be unrelated to information about the individual which means that it cannot be derived from that information nor can it otherwise be capable of being translated so as to identify the individual. Typically, this means that re-identification keys must not be derived from PHI and must be separately and securely stored to prevent simultaneous access to the de-identified data and the re-identification dataset that would allow render the DIHI as PHI. It is also worth noting that the chance of present or future re-identification of DIHI may not be deemed to be zero given unknown advances in technology or data science. Therefore, in some embodiments, the goal for de-identification is that the risk of re-identification be “very small.”

While de-identification of sensitive information has many potential advantages, such as those described above, there are also disadvantages to removing all personal identifiers from PHI. One obvious disadvantage is the loss of specific identifiers that would enable parties to access that specific data. For example, removal of a patient's name, date of birth and medical record number from a health record could make it impossible for a provider or patient to find that patient-specific record with standard search methods since the patient's identifiers would no longer be associated with the data. Another disadvantage of removing individual identifiers from healthcare data relates is the loss of temporal information and dates, such as dates of hospitalizations, procedures, or dates of clinic visits. As described earlier, temporal analysis of health care data may be useful or necessary when evaluating a patient's disease progression, treatment response or other facets of their healthcare history. Loss of temporal information, such as dates and times, from a serial set of healthcare data can disrupt the analysis of a patient's disease progression, treatment response or other serial aspects of a patient's healthcare. For example, given two sets of health data taken at different times and showing better and worse health states respectively, the prognosis for the patient would be quite different if the worse disease state occurred on the earlier date (meaning improvement in the health of the patient) than if the worse disease state occurred on the later date (meaning overall worsening of the patient's health state). In this example, removal of dates from these two records would prevent a provider from correctly determining whether the patient was worsening or improving. Many other negative consequences of removal of dates from health records also exist.

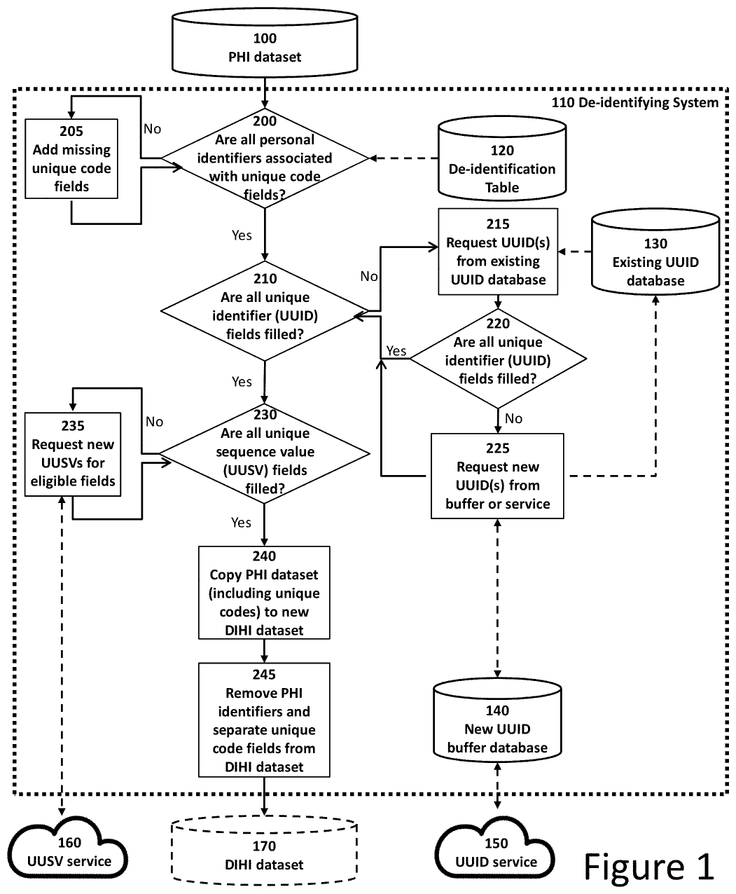

With reference to FIG. 1, there is illustrated a flowchart depicting one embodiment of de-identification system 110. In this embodiment, de-identifying system 110 accepts PHI dataset 100 as an input and produces both an updated form of PHI dataset 100 and DIHI dataset 170 as an output. A principle of this embodiment is that de-identifying system 110 preserves the integrity, temporal order and unique assignment of PHI dataset 100 in the output that it generates which is DIHI dataset 170. In some embodiments, PHI dataset 100 originates from an ophthalmic diagnostic device such as an optical coherence tomography device or an automated eye examination device. De-identifying system 110 can be a computer, server or group of computers or servers that 1) runs software to de-identify personally identifiable data, such as health data, using unique codes and 2) is connected electronically to a unique code service or services such as via a network, phone or other electronic connection. Unique codes are comprised of universally unique identifiers, hereafter also referred to as unique identifiers or UUIDs, and universally unique sequence values, hereafter also referred to as unique sequence values or UUSVs. In various embodiments, UUIDs and UUSVs can both be used in the same healthcare data de-identification process. In some embodiments, only one of UUIDs or UUSVs is used to de-identify a healthcare record without the other.

For the purposes of this discussion, a universally unique identifier or UUID is data, such as a number, an alphanumeric string, a bar code, a QR code or other type of code, that is supplied by a UUID issuing service 150. UUIDs are comprised of at least two sub-types: centralized and non-centralized. In various embodiments, these types may be used interchangeably while in other embodiments, one type of UIID or the other may be used. The main difference between the two types of UUIDs is that non-centralized UUIDs are issued by non-centralized issuing services which means that they are not guaranteed to be unique. In contrast, centralized UUIDs are guaranteed to be unique since they are issued by a single central service that monitors and checks all UUIDs that it produces. A non-centralized UUID issued by a non-centralized service should satisfy at least two criteria: 1) it should be created in a large enough dataspace such that the chance of issuing a duplicate UUID is very small and 2) it should not be derived from or related to information related to or about the individual and cannot otherwise be capable of being translated so as to identify the individual. The dataspace of a UUID refers to the number of possible permutations of values for the UUID given the size and/or length of the UUID. For example, the dataspace for a standard 36 character HEX-based UUID may be any 128-bit number. A centralized UUID issued by a centralized service is similar to a UUID issued by a non-centralized service except that it is checked for uniqueness before being issued. A centralized UUID should satisfy at least two criteria: 1) it should not have been issued previously nor should it be issued again at a future time and 2) it should not be derived from or related to information related to or about the individual and cannot otherwise be capable of being translated so as to identify the individual.

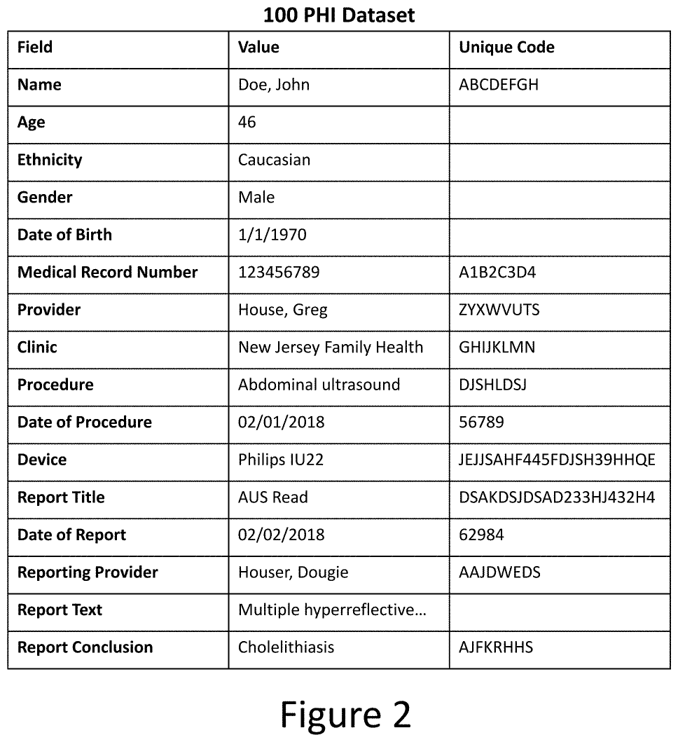

An important advantage of a centralized UUID that is guaranteed to be unique is that it can be created from a smaller dataspace than a non-centralized UUID. For example, a standard non-centralized UUID may be 24, 36 or even 48 characters in length because it must be generated from a large enough dataspace, such as 128-bit data, to reduce the chance of issuing the same UUID twice. In contrast, a centralized UUID might only consist of 4, 8 or 10 characters and be created from a much smaller data space than a 128-bit UUID since the size of the data space for a centralized UUID isn't used to reduce the chance of duplicate publication. FIG. 2 shows examples of centralized UUIDs in the column labeled Unique Code. In cases where individuals are asked to remember or manually record a UUID, the shorter length of a centralized UUID is advantageous since it is easier to remember or write down. In some embodiments, use of a smaller dataspace for centralized UUIDs might also be advantageous if the central UUID service searches the data space for duplicates prior to publication. Keeping the dataspace small can make this search faster and more efficient.

Both centralized and non-centralized UUIDs have many potential uses in the de-identification of personally-identifiable health data. FIG. 2 shows how unique codes can be associated with personally-identifiable healthcare data fields prior to removal of the individually-identifiable data to uniquely but not personally identify this data after de-identification. In some embodiments, these unique code fields can be created at the same time that the PHI dataset 100 is created. In other embodiments, these unique code fields can be added after creation of the PHI dataset 100. FIG. 2 shows how centralized UUIDs, one type of unique code defined above, can be used to identify an individual, such as a patient in a medical clinic, in a de-identified record since the UUID cannot be reversed to deduce the PHI of that individual. Non-centralized UUIDs can also be used to identify individual. Either type of UUID can also be used to identify a medical clinic itself or a staff member, including the provider, in a clinic. Either type of UUID can be used to identify a service, such as an exam or a test. Both types of UUIDs can also be used to identify a diagnostic device, surgical device, computer, server or any other discrete component used in the provision of health care. Many other potential uses of UUIDs exist.

As stated above, centralized and non-centralized UUIDs are produced by a UUID service 150 which is an electronic service and may be a single entity or multiple different entities. There are many reasons why UUID service 150 must be an electronic service and cannot be accomplished through manual labor. In the case of a centralized service that must search all prior UUIDs to ensure uniqueness, it would be too time-consuming and impractical for a human worker to guarantee the uniqueness of centralized UUIDs by searching through millions or hundreds of millions of prior UUIDs each time a new UUID must be generated. In the case of non-centralized UUIDs which are generated from extremely large number spaces, it would be time-consuming and impractical for a human being to perform the calculations necessary to generate a pseudorandom number in this dataspace. It would also be time-consuming and inaccurate to convey the long result to the requesting service if it were done manually. For a UUID service that only needs to generate one million UUIDs per year, which is a small fraction of the UUIDs that may be required of this system, the service would need to generate ten UUIDs per second during business hours. This is much faster than a human worker can operate. Therefore, the only practical embodiment for UUID service 150 is as an electronic service.

UUID service 150 may exist within de-identifying system 110 or exist outside of but be electronically connected to de-identifying system 110. In some embodiments where UUID service 150 exists outside of de-identifying system 110, UUID service 150 can exist in a location that is remote from de-identifying system 110 but electronically connected, such as a website or web service, and more easily accessible to other de-identifying clients. UUIDs may be stored in a variety of databases, such as existing UUID database 130 and new UUID buffer database 140, and these databases may exist within de-identifying system 110 or exist outside of but be electronically connected to de-identifying system 110. In various embodiments, the location of existing UUID database 130 can be intentionally separated from the location of DIHI dataset 170 since existing UUID database 130 acts as a re-identification key for DIHI dataset 170. As stated previously, co-location of de-identified data with its re-identification key may constitute a security risk in the event that that location's security is compromised and the DIHI data can then be rendered back to PHI data.

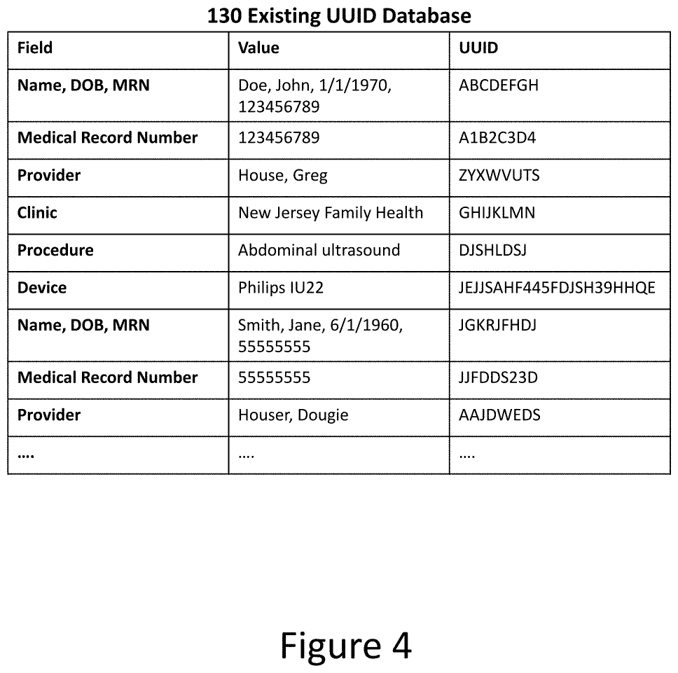

As shown in FIG. 4, existing UUID database 130 is comprised of previously-generated UUIDs stored in association with the PHI data that they are meant to replace or modify in de-identified dataset 170. Existing UUID database 130 contains only UUIDs and does not contain UUSVs since UUSVs are requested from the central service on the date that is to be replaced in the healthcare record and are not typically stored or buffered for future use. Existing UUID database 130 effectively acts as a look-up table that establishes a one-to-one relationship between a specific PHI dataset field value and the UUID that will be used to modify it in de-identified dataset 170. By searching existing UUID database 130 prior to requesting a new UUID, de-identifying system 110 can use an existing UUID value or values for PHI field values, such as a specific patient's name or medical procedure, that have been encountered previously. For the sake of clarity, zero, one, or more than one UUID can be associated with data found through a search query of existing UUID database 130. If more than one UUID is associated with the data found using the search query, such as multiple UUIDs for a single patient or clinic, de-identifying system 110 can be configured to use all or only some of the discovered UUIDs. If de-identifying system 110 does not find a matching PHI field value in existing UUID database 130, it can request a new UUID from new UUID buffer database 140.

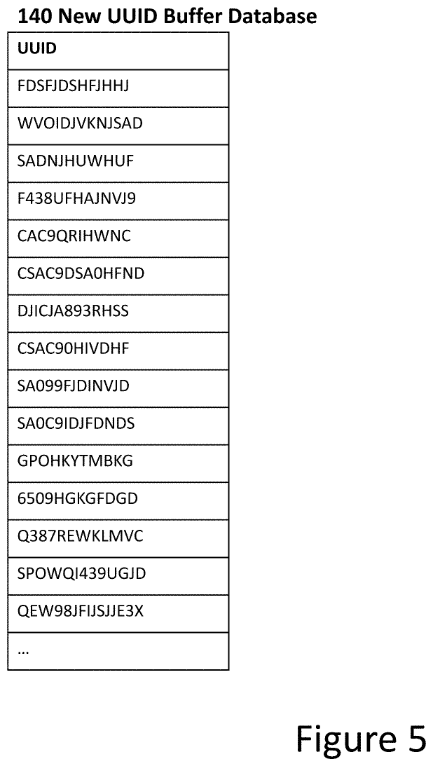

As shown in FIG. 5, new UUID buffer database 140 is comprised of a list of unassigned UUIDs received from UUID service 150. In the embodiment shown in FIG. 1, de-identifying system 110 requests new UUIDs from new UUID buffer database 140 at step 225 which occurs after de-identifying system 110 has already checked existing UUID database 130 to confirm that a UUID does not already exist for this particular data value. Once received, the new UUID and associated PHI data are added to existing UUID database 130 for recall when the same PHI data is encountered in the future. The UUIDs in new UUID buffer database 140 are created after requests to UUID service 150 for single UUIDs or sets or blocks of UUIDs initiated either by de-identifying system 110 or by new UUID buffer database 140 itself. In some embodiments, new UUID buffer database 140 or de-identifying system 110 can be configured to automatically request new UUIDs when the total number of available UUIDs in new UUID buffer database 140 drops below a preset threshold such as 10, 100, 500 or 1,000. In the event that new UUID buffer database 140 does not have any UUIDs in its database, de-identifying system 110 can be configured to request new UUIDs directly from UUID service 150. An advantage of de-identifying system 110 requesting new UUIDs from new UUID buffer database 140 instead of from UUID service 150 is that de-identifying system 110 can continue to operate when communication with UUID service 150 is interrupted or when UUID service 150 is offline or otherwise unresponsive. This buffering system will only work as long as new UUID buffer database 140 contains UUIDs and/or UUID service 150 is available and able to provide new UUIDs upon request.

For the purposes of this disclosure, a universally unique sequence value or UUSV is an ordered value, such as a numeric value or value from another type of ordered set such as an alphabetic set, that 1) is larger (or sorted later in a non-numeric set) than any UUSV previously issued, 2) is smaller (or sorted earlier in a non-numeric set) than any future issued UUSV, and 3) is not derived from or related to information about the individual and cannot otherwise be capable of being translated so as to identify the individual or the date or time of the request. In some embodiments, UUSV service 160 can be configured to reset the UUSV sequence to a starting value, such as zero, at the beginning of each calendar year to reduce the maximum size of the UUSV values that are generated. This embodiment is only possible when the year portion of a date field is not considered to be PHI and can be preserved in a de-identified dataset. As an example of this embodiment, de-identifying system 100 would replace a complete date value such as Jun. 1, 2019 with only the year value (2019) and augment that year value with a UUSV that obeys the ordering rules specified previously but applied only to calendar year 2019.

As with UUIDs, UUSVs must be produced by an electronic service and cannot be produced through manual labor. The main reason for this requirement is that millions of UUSVs may be requested by de-identifying services during business hours in a given year. Using a conservative estimate of 1 million UUSV requests per year received during business hours, the average response time for a UUSV result would need to be 125 milliseconds with a peak demand response time of less than that. As with UUIDs, UUSV service 160 may need to be capable of providing a UUSV response quickly, such as within 10 milliseconds, 100 milliseconds, or 1000 milliseconds. It is even more imperative that UUSVs be generated quickly since UUSVs cannot be buffered the way that UUIDs can. In fact, any delay in UUSV generation may be passed onto to the UUSV service's clients which, if designed to wait for a UUSV result before proceeding with de-identification, will substantially degrade the performance of the UUSV's services clients.

In some embodiments, UUSVs can be created by UUSV service 160 at the same time that the PHI dataset 100 is created. In other embodiments, UUSV fields can be added and filled after creation of the PHI dataset 100. In still other embodiments, UUSV fields can be created at one point in time but remain empty until being filled with UUSV data at a later point in time. UUSV service 160 may exist within de-identifying system 110 or exist outside of but be electronically connected to de-identifying system 110. In some embodiments where UUSV service 160 exists outside of de-identifying system 110, UUSV service 160 can exist in a location that is remote from de-identifying system 110 but electronically connected, such as via a website or web service, and more easily accessible to other de-identifying clients. In many embodiments, UUSVs are issued by a UUSV service 160 that is configured to issue UUSVs to multiple de-identifying systems 110. In this context, these multiple de-identifying systems 110 can also be referred to as clients or customers of UUSV service 160. One advantage of this arrangement is that all de-identifying system clients that share the same UUSV service 160 can maintain the proper order of their recorded health events even when their data is de-identified and combined at a future time. In some embodiments, UUSV service 160 provides publicly-visible unique sequence values. In other embodiments, values passed from UUSV service 160 to its de-identifying system clients are private or otherwise secured so as not to be visible to non-client entities. This configuration is advantageous as it prevents outside entities from requesting UUSVs and developing a table of unique sequence values in association with real date values for the purposes of decoding UUSV date fields. While de-identifying system clients could do this same thing, it is unlikely that they would choose to do this given their designed purpose of de-identifying healthcare data.

UUSVs are used to replace or otherwise augment dates, such as any combination of a month, day and year, and/or times, such as any combination of hours, minutes, seconds or milliseconds, in PHI datasets during the creation of a de-identified dataset. In some embodiments, a UUSV can replace an entire date and time stamp in a PHI dataset. In other embodiments, a UUSV can be used to replace only the day and month components of a date without replacing the year or the time. Other variations on partial date and time substitutions by UUSVs are possible. In many embodiments, UUSVs are requested on the date that is to be stored in the DIHI data. Failure to request a UUSV on the same date as the date of record does not negate the benefit of the UUSV but it does reduce the precision of the temporal order recorded by the UUSV. FIG. 2 shows examples of UUSVs associated with two different dates in a medical record. Note that the UUSV associated with the later date is numerically larger than the UUSV associated with the earlier date since UUSVs increase in their ordinal value over time. In some embodiments, sequential UUSV values are offset from each other by preset increment amounts that create predictable series such as 1, 2, 3 or 3, 5, 7 or 100, 200, 300. In other embodiments, sequential UUSV values are offset from each other by arbitrary increment amounts that create less predictable series such as 1, 17, 19 and 100, 719, 801 and 500, 501, 900. An advantage of the latter embodiment is that it obscures the number of UUSV requests that occur between any two UUSV values. In many embodiments, the starting value, such as 1, 3, 100, or 500, used to calculate subsequent sequence values by UUSV service 160 is configurable either by UUSV service 160, by a consensus of UUSV service's clients, or by a lead client of UUSV service 160.

In many embodiments, UUSVs are requested from UUSV service 160 and issued on the date of a service or event. In some embodiments, UUSVs are issued both on the date and at the time of an event to further increase the precision of the serial ordering capability of the UUSV. The main purpose of a UUSV is, in fact, to sort or enable ordering of de-identified records that have had their dates removed and are subsequently compared or combined. For example, if the dates of two or more services are removed in the creation of a de-identified health information database, the order of occurrence of these services, such as which service occurred first, can no longer be determined. In the case of a de-identified dataset that includes UUSVs issued on the date of each service, the order of the services in the de-identified database can still be determined by comparing the UUSVs associated with the two services since the UUSV is an ordered value and the UUSV of the earlier service will have a lower UUSV value than that of the later service. It is important to note that a UUSV still satisfies the requirements for de-identified data since it cannot be used to deduce any identifiable information about the services such as a date associated with the service.

In some embodiments, the first step in the process of de-identifying individually-identifiable health information occurs when PHI dataset 100 is flagged for de-identification and transferred, such as electronically, via a network interface, programmatically by reference, or programmatically by value, to de-identifying system 110. PHI dataset 100 can be comprised of many pieces of data related to a single individual or related to multiple individuals. For example, a record of a clinic visit created for a specific patient can constitute PHI data. Records of multiple procedures on one or more than one patient can also constitute a PHI dataset. Many other types of individually-identified health information can comprise PHI dataset 100.

In many embodiments of the present disclosure, the next step in de-identifying a PHI dataset after identification and transfer of PHI dataset 100 to de-identifying system 110 is for de-identifying system 110 to confirm that unique codes are available for all PHI identifiers before they are removed from PHI dataset 100. If PHI identifiers are removed before unique codes are available in the dataset, the unique codes can only be associated with de-identified data. Association of UUIDs with de-identified data instead of PHI data greatly decreases the usefulness of the unique coding de-identification system since, at the very least, it negates the utility of existing UUID database 130 as a re-identification key.

In FIG. 1, steps 200 through 235 show one embodiment of a process used by de-identifying system 110 to confirm that unique codes are available prior to removal of PHI identifiers. In step 200, de-identifying system 110 checks to see if PHI identifiers in PHI dataset 100 are properly associated with unique code fields. For example, if PHI dataset 100 contained a field for patient name but not field for a UUID for the patient's name, de-identifying system 110 would identify that unique code field as one that needed to be added. In the embodiment in FIG. 1, de-identifying system 110 loops through all available PHI dataset 100 field names and compares the field names to pre-existing information about which field names in PHI dataset 100 represent PHI identifiers. In some embodiments, this information could be encoded in PHI dataset 100 already. In other embodiments, the process of identifying which fields in PHI dataset 100 comprise PHI identifiers could be programmed into the software code of de-identifying system 110 in the form of rules to be applied to PHI dataset 100. In the embodiment shown in FIG. 1, PHI identifier information is provided to de-identifying system 110 in the form of de-identification table 120. Many other embodiments are possible.

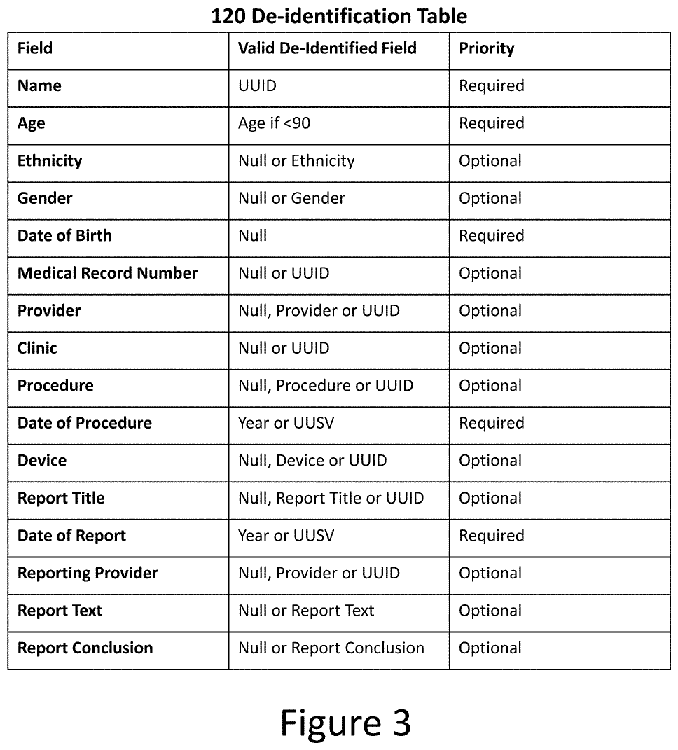

FIG. 3 shows an example of de-identification table 120 which, in various embodiments, may be comprised of records containing 1) field names from PHI dataset 100, 2) the de-identification disposition of those fields, such as whether to remove, nullify, replace or leave the data contained therein unchanged when copying data from PHI dataset 100 to DIHI dataset 170, and 3) the importance of making those changes to the PHI dataset 100 field values. As stated above, in various embodiments, information from de-identification table 120 could instead be encoded into PHI dataset 100 to obviate the need for a separate look-up table. The information contained in de-identification table 120 could also be encoded in the software of de-identifying system 110. In various embodiments, more fields or fewer fields can be used to store the information in de-identification table 120 which indicates how each PHI identifying field value in PHI dataset 100 is treated during the process of de-identification. In some embodiments, a field priority can be marked as “optional” to indicate to de-identifying system 110 that the field does not need to be filled prior to de-identification even if the unique code field is found to be empty in steps 210 or 230.

In the embodiment shown in FIG. 1 at step 200, de-identifying system 110 surveys fields in PHI dataset 100, such as name, medical record number, and clinic, and checks these field names against field names (shown in the first column of the table in FIG. 3) and associated unique codes (shown in the second column of the table in FIG. 3) in de-identification table 120. For PHI dataset 100 fields that do not already have pre-existing associated unique code fields available in PHI dataset 100, de-identifying system 110 will execute step 205 to insert a blank unique code field into PHI dataset 100 according to the disposition and priority requirements from de-identification table 120. For PHI dataset 100 fields that already have pre-existing unique code fields indicated as either required or optional by de-identification table 120, de-identifying system 110 skips them and moves onto the next field in PHI dataset 100 until it reaches the end of the dataset. If de-identifying system 110 does not encounter any PHI identifier fields in PHI dataset 100 that are flagged by de-identification table 120 for de-identification, de-identifying system 110 will proceed to the next step 210.

In the embodiment shown in FIG. 1, after de-identifying system 110 has confirmed that unique code fields exist for PHI identifier fields in PHI dataset 100 according to the information in de-identification table 120, de-identifying system 110 moves on to step 210 to confirm that all unique identifier fields are populated with unique identifiers. If de-identifying system 110 encounters an empty unique identifier field when looping at step 210 through all available unique code fields in PHI dataset 100, de-identifying system 110 executes steps 215, 220 and possibly 225 for each of those fields. In step 215, de-identifying system 110 compares the PHI identifier field value to values in existing UUID database 130 to determine if a UUID code has already been associated with that PHI identifier field value. If a match is encountered for a particular PHI identifier field value, such as “House, Greg” from the FIG. 4 example, the UUID associated with that value is copied to the empty unique code field in PHI dataset 100 per the instructions established by de-identification table 120 and de-identifying system 110 returns to the loop at step 210 to check for additional empty unique identifier fields in PHI dataset 100. If no match for the PHI identifier field value is found in existing UUID database 130, de-identifying system 110 moves on to step 225 to request a new UUID from new UUID buffer database 140. If a UUID is returned from new UUID buffer database 140 to de-identifying system 110, that pair of values (PHI identifier field value and associated UUID) are passed to existing UUID database 130 to be stored for future reference and de-identifying system 110 returns to the loop at step 210 to check for additional empty unique identifier fields in PHI dataset 100. If no UUID is returned from new UUID buffer database 140 to de-identifying system 110, de-identifying system 110 can be configured to request a UUID directly from UUID service 150. If no communication can be established between de-identifying system 110 and UUID service 150, de-identifying system 110 can be configured to issue an error or issue a temporary UUID which can be used for de-identification purposes until it can be replaced with a universal UUID from a UUID service 150, such as when communication with UUID service 150 is re-established and a new block of UUIDs can be requested from UUID service 150. In this embodiment, temporary UUIDs would be stored in a database similar to or exactly comprised of the new UUID buffer database 140.

In embodiments of the present disclosure, once de-identifying system 110 has confirmed that unique identifier fields are filled for PHI identifiers in PHI dataset 100 identified by de-identification table 120, de-identifying system 110 proceeds to step 230 to confirm that unique sequence value fields are populated in PHI dataset 100. If de-identifying system 110 encounters an empty unique sequence value field when looping at step 230 through all fields in PHI dataset 100 indicated as requiring a UUSV by de-identification table 130, de-identifying system 110 executes step 235 to request a new UUSV directly from UUSV service 160. After the newly received UUSV is stored in PHI dataset 100, de-identifying system 110 returns to the loop at step 230 to confirm that all eligible unique sequence value fields are populated in PHI dataset 100. If de-identifying system 110 does not receive a UUSV from or cannot communicate with UUSV service 160 and the PHI field is designated as ‘required’ in de-identification table 120, de-identifying system 110 can be configured to generate an error or to proceed with de-identification in the absence of a UUSV for that specific PHI field.

Once de-identifying system 110 has confirmed that all eligible unique identifier fields, including both UUID and UUS V fields, are filled for the PHI identifiers in PHI dataset 100 identified as required by de-identification table 120, de-identifying system 110 proceeds to step 240 to copy data from PHI dataset 100 to a new data structure that will become de-identified dataset 170. In step 245 of the embodiment shown in FIG. 1, de-identifying system 110 loops through all fields in this new data structure and uses information from de-identification table 120 to determine the disposition of each PHI field in the de-identified dataset. For example, some values in PHI dataset 100 will be left unmodified if they are not designated as PHI identifiers. Some values in PHI dataset 100 will be modified, such as by being replaced by an associated unique code as indicated in de-identification table 120. Some values in PHI dataset 100 will be deleted entirely leaving their fields empty. Other modifications of PHI dataset 100 fields are possible. Other orders for steps 240 and 245 are also possible.

The data created upon completion of step 245 by de-identifying system 110 is de-identified dataset 170. At this stage, it is important to note that PHI dataset 100 may also have been modified during this process with updated unique codes. Therefore, the outputs from de-identifying system 110 include both de-identified health information dataset 170 and updated PHI dataset 100. De-identifying system 110 may transfer DIHI dataset 170 across a network to another server or multiple other servers, to an attached storage method, such as a hard disk drive, flash memory drive, or other portable media, to a mobile device via a wireless connection such as a Bluetooth connection, a WiFi connection, or via SMS, or maintain a copy of DIHI dataset 170 in local storage. Various combinations of these methods of transfer and storage are possible.

In embodiments where DIHI dataset 170 is received by another computer, DIHI dataset 170 can be added to a database containing other de-identified datasets, hereafter referred to as a central repository of de-identified health information. This central repository of de-identified health information can exist as a single database, multiple databases, a database managed by a distributed file system or other means of storing and retrieving large amounts of data. Access to this central repository can be managed through distribution, or lack of distribution, of the UUIDs to various parties. For instance, the UUID associated with an individual patient may be given to the patient in printed form, such as on a piece of paper or a pre-printed card, but not given to anyone else. The UUID may also be distributed in electronic form, such as on a USB key, via email or via SMS message such as to other healthcare providers who take care of that patient. Various other methods of distributing UUIDs to parties such as patients and providers are also possible.

In embodiments in which UUIDs are supplied to patients, UUIDs can enable patients to access their de-identified health data from a central repository of data using the UUIDs. For example, a central repository could be comprised of a database of DIHI and software configured to enable querying of the DIHI database to find specific records within the database. This software could be used by a patient to access their specific de-identified data. In the simplest form, the patient could use a Web browser to access the software and data, such as by inputting an easily remembered Web URL. Once at the website running the aforementioned software, hereafter referred to as a Patient Web Portal or Patient Portal, the patient could input their UUID into a search field in the software. Once the query executed by the central repository retrieves the data associated with that UUID, the patient Web portal could be configured to allow the user to view, download or print the data. A slightly higher level of security, through stricter access control, could be achieved by the patient Web portal by requiring a response, such as a CAPTCHA response, that could decrease the likelihood that automated software, such as a Web bot, could access a patient's record by attempting to log in with multiple random UUIDs. A higher level of security could also be achieved after entry of a UUID by requesting authentication with durable, de-identified values associated with that UUID, such as the patient's gender at birth, race, age (if less than 90 years old), state of birth, and country of birth. Other durable items could be used for the purpose of authentication as long as they do not qualify as PHI and are unlikely to change throughout the life of a patient.

After a user's query to view a record associated with a UUID has been accepted by the patient Web portal, a number of features can be configured to be available to the user on the patient Web portal. The simplest feature is to view, download and print DIHI, such as exam findings or results from a single visit or multiple encounters with a provider, contained therein. An important aspect of the patient Web portal is that it can be configured to allow a patient to access DIHI from multiple examinations and from multiple providers in a single location. For example, if a patient sees multiple providers for care and those multiple providers upload DIHI to the central repository, the patient can access DIHI data from all of those providers in one location—a capability that would not be possible in most healthcare settings where providers typically use different means of storing and retrieving health information.

In various embodiments, the patient Web portal can also be configured to sort records temporally, even in the absence of a date of service, using the UUSV included in the DIHI dataset. As described previous, UUSVs are generated sequentially and thus, the order of DIHI can be deduced even if the exact time intervals cannot be recovered. For example, if a patient sees a doctor in February of a given year and then sees another doctor in March of that same year, the patient Web portal would be able to deduce the order of the exams since the UUSV for the first exam would be less (or occur earlier in a list of items) than the UUSV for the second exam. In this way, even exams with dates removed from them as part of the de-identification process can be ordered properly in the patient Web portal.

A UUID associated with a patient can also be used to allow a patient to control which providers are allowed to have access to their data. For example, if a patient gives their UUID to a provider during a clinic encounter, that UUID can be used by that provider to access prior health information for the patient by querying the database of DIHI on the central repository, such as via a network connection. The provided UUID can also be used by the provider to add health information from that new encounter to the patient's DIHI in the central repository by uploading de-identified healthcare data to a central repository for integration with other data associated with that UUID. In another situation, a patient may choose not to share their UUID with a provider. In that case, the provider can issue another UUID to the patient thereby allowing the patient to keep those two sets of DIHI separate. If the patient chooses to unite the two DIHI datasets at a later date, the patient Web portal could be configured with a function to enable merging of different UUIDs such as through a list of shared UUIDs for a single individual. That list of shared UUIDs does not need to exist only in the central data repository and can be transmitted to all clinic UUIDs associated with that patient UUID. Items in the list of shared UUIDs for an individual can be edited such that UUIDs can be entered or removed. This allows a patient to have control over how their DIHI is shared among their providers. Patients may also choose to remove previously shared UUIDs, cutting off access to the dataset.

Another method of control that the patient Web portal can provide to a patient is to allow patients to share DIHI with providers by name. In many areas of the world, the name of a patient's provider is not considered PHI. Therefore, a provider's name can be included in a patient's DIHI, can be displayed to the patient through the patient Web portal, and can be part of database queries formulated by the patient. Furthermore, a patient may choose to share some portions of their DIHI with some providers but not others. This additional “provider name”-based control of DIHI access gives even more control to patients using the patient Web portal.

Another capability of the patient Web portal is the ability for a user to create a shareable link to a specific group or piece of data. For instance, a user could select a single clinic visit encounter from the patient Web portal and generate a shareable link to this DIHI. This link could be sent, such as via email or text message, to another individual so that they could access the data identified by the patient. This could be useful, for instance, in the case of a patient who would like to share specific visit information with a provider who may not have participated in that visit. Alternatively, the link could be sent to a third party service, such as a second opinion diagnostic service, to gain additional information about the health status of the patient demonstrated in that DIHI. Additional information, such as patient education materials, second opinion diagnostic services, and differential diagnostic services, could also be made available to users of the patient Web portal.

Another use of a patient UUID is to allow different clinic sites or different providers to synchronize health information for a patient. If, for example, a patient sees two providers and uses the same patient UUID for those encounters, the local databases at each provider's clinic could be synchronized to contain the same DIHI for the patient. For example, if a patient sees a first provider in February and then sees a second provider in March, the provider at the time of the March visit could generate a query using software executed by the central repository to search for DIHI associated with the UUID provided by the patient to the provider. If the February DIHI associated with that UUID is found by that query and does not already exist in the database in the March clinic, that DIHI could be downloaded to the March clinic's database in the form of DIHI. In that way, the March provider could automatically have access to prior health information for the patient—dependent entirely on the patient's desire to share the UUID used for the February visit. Taking it one step further, when the patient in question returns to see the first provider at an even later visit, a query from the original provider's office could be used to retrieve data from the March visit and the resultant data from the April visit could similarly be retrieved by the March clinic's database to fully synchronize the health data at each provider's clinic. It is important to note that once these DIHI datasets are downloaded to the respective clinic computers, they can be re-identified by merging the DIHI record into the local database after authenticating the DIHI record with identifiers discussed previously, such as gender at birth and age, contained in the clinic computer's database.

Transfer of this DIHI data from the clinic computer to the central repository or from the central repository to the clinic computer may need to be managed so as not to negatively impact the Internet bandwidth of the clinic computer. For example, data uploads and downloads could be organized hierarchically such that the smallest data, such a text or numerical data, could be downloaded or uploaded first followed by larger datasets such as images and video. This could be advantageous in clinics with limited download or upload bandwidth to allow them to quickly access DIHI data from the central repository. Another method of optimization for transfer of DIHI data is to schedule portions or all of the data transfers for hours with lower bandwidth usage, such as overnight. For example, if a clinic computer was scheduled to upload a new batch of DIHI from patient encounters during the day, the smaller data, such as text or numerical data, could be uploaded during clinic hours while the larger data, such as images and video, could be uploaded after the clinic closes when the bandwidth needs are reduced. Further, in clinics with intermittent or limited Internet connectivity, portions of DIHI data could be flagged as high priority such that the majority of the bandwidth is devoted to transfer of that data. DIHI data could also be queued for transfer in clinics with intermittent Internet connectivity such that all DIHI data transfer occurs when a working Internet connection is established.

In some embodiments, a central repository of DIHI data aggregated from numerous healthcare events, such as hundreds, thousands or millions, can be analyzed to determine normal ranges for values contained within the DIHI data. For example, color vision measurements from DIHI datasets from numerous eye exams could be aggregated into a single dataset for analysis. If the health status of each patient contributing data to that dataset is not known, the dataset can be referred to as a mixed dataset since it presumably comprises color vision values from both normal and abnormal individuals. If the health status of a group of patients is known to be normal for the dataset value being studied, the resultant dataset could be referred to as a normal dataset. Similarly, if the health status for a group of patients is known to be abnormal for the dataset value being studied, the resultant dataset could be referred to as an abnormal dataset.

The aforementioned normal, abnormal and mixed datasets can be further segmented, or split into subgroups, prior to analysis. Commonly used subgroups include but are not limited to: subdivisions based on gender, subdivisions based on ethnicity, and subdivisions based on age. Selection of the proper subgroup segmentation can be based on statistical measures, such as sample variance, biological or pathophysiological knowledge, such as the fact that older patients are more likely to have cataracts, or based on random search.

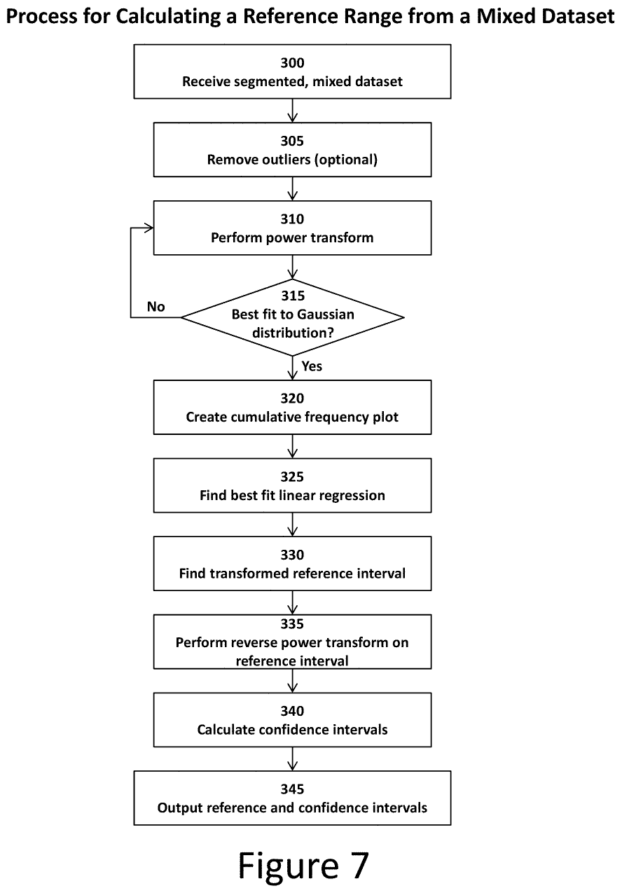

Discovery of the values in the aforementioned datasets that lie at the boundary between normal and abnormal measurements, the so-called reference intervals, can be performed automatically using techniques, such as those outlined in Katayev et al (Katayev et al. “Establishing Reference Intervals for Clinical Laboratory Test Results. Is There a Better way?” Am J Clin Pathol. 2010; 133:180-186. And Katayev et al. “Reference Intervals Data Mining. No Longer a Probability Paper Method.” Am J Clin Pathol January 2015; 143:134-142.). FIG. 7 demonstrates how a software system can be configured to accept a mixed, segmented dataset as an input, discover the reference interval limits, such as 2.5% and 97.5% of the normal distribution, for the values contained in the segmented dataset, and output those values along with ranges of uncertainty. An important limitation of the disclosed approach is that it may not work well for mixed datasets where the number of abnormal values exceeds the number of normal values. The performance of this approach is also expected to decrease as the total number of values in the dataset decreases and as the natural distribution of the data deviates markedly from a normal or Gaussian distribution.

According to the embodiment shown in FIG. 7, the process of discovering the reference intervals for a mixed dataset begins with receipt of the mixed dataset by a software program capable of executing software code to find the reference intervals of the mixed dataset. The next step in the process, which is optional, is to remove outliers from the mixed dataset such as by iteratively applying Chauvenet's criterion to the data as described by Katayev. After data outliers have optionally been removed, the first major step in the process is to iteratively find the power transform, such as a Box-Cox transform, that transforms the mixed dataset into a distribution that most closely mimics a Gaussian distribution. Once the transform that most closely approximates a Gaussian distribution has been found, the transformed data can be sorted into a cumulative frequency distribution by sorting the values from lowest to highest. In this cumulative frequency distribution, the ith location in the dataset corresponds to the ith/nth percentile within the dataset where n represents the total number of values in the dataset. A cumulative frequency plot can be created by plotting the percentile value on the abscissa and the sorted value on the ordinate axis for every value in the dataset. A best-fit linear regression can be performed on this plot to find the longest linear fit that satisfies a maximum deviation criteria such as the maximum residual error criteria proposed by Katayev or a maximum deviation criteria based on the expected standard deviation of the points between reference intervals on an ideal Gaussian curve. Once the best linear fit has been found, the most extreme points wherein all intervening points lie within the aforementioned maximum deviation criteria define the reference intervals of the mixed dataset. Prior to outputting those intervals, the power transform performed at the early stages of the analysis must be reversed and confidence intervals can be calculated. During the final stage of the analysis, the system can output the reference and confidence intervals.

In various embodiments, the reference intervals that are output by the system described above can be transmitted to the same systems that contributed the de-identified data to the central repository and enabled the development of those reference intervals. In fact, it is logical that these reference intervals would apply most directly to the data used to generate them, so the systems contributing de-identified healthcare data would be most appropriate consumers of the reference intervals. These systems, which contain both de-identified and individually-identified healthcare data, can apply these reference intervals to healthcare datasets when outputting or showing that data to healthcare providers. The advantage to this embodiment is that the healthcare providers can receive guidance on which values are abnormal, or outside of the normal reference range values, and which values are normal or within the normal reference range values. Outputted results can be shown to providers in numerous ways such as with histograms of where particular values fall on the distributions for those values see in the data, color coding of results to call attention to abnormal results (such as coloring them red), or using a formatted approach similar to a standard blood chemistry report where abnormal results are spatially offset from normal results.

When applied to ophthalmic diagnostic data, a de-identification system in communication with an ophthalmic diagnostic device can be configured to de-identify eye examination data from that ophthalmic diagnostic device. That de-identified data, which can include one measurement or numerous different measurements from the eye, can be transmitted to a central repository of de-identified data where it can be combined with de-identified data from multiple other locations and other times captured with the same ophthalmic diagnostic device. Using this central repository, the system described previously can automatically calculate the normal range of values for every measurement in the dataset and return these reference range values to all systems that contributed data to the central repository. Those systems can then apply those reference ranges to the measurement data, both individually-identified and de-identified, when the data is shown to the healthcare providers so that those healthcare providers can quickly and easily interpret the disease state of an individual by seeing which, if any, of the numerous measurements taken are abnormal.

### Automated Eye Examination

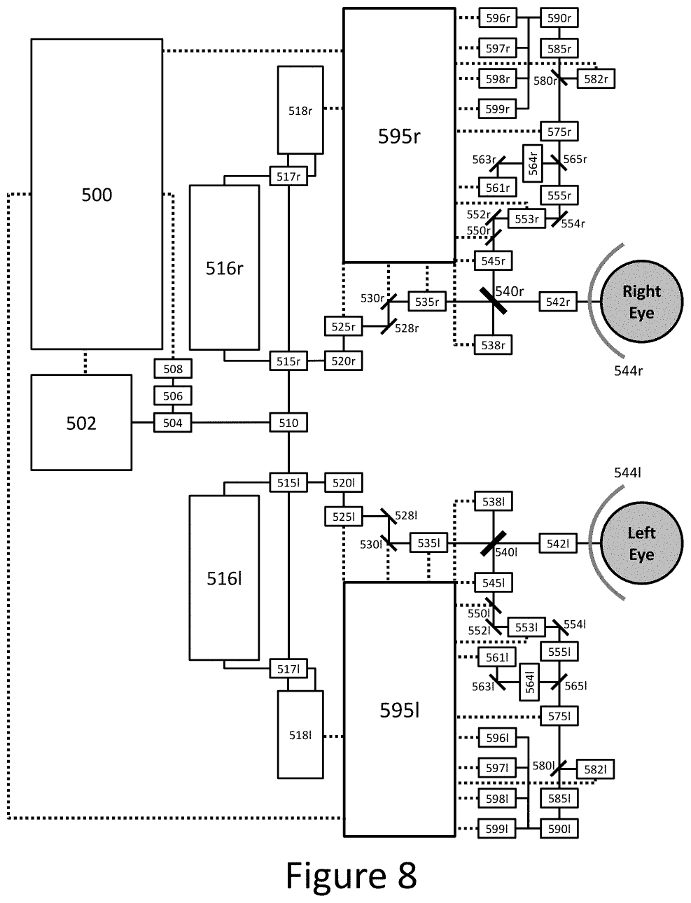

FIG. 8 shows an example of a binocular eye examination system based on optical coherence tomography. The control electronics 500 may be comprised of the main electronics, processors, and logic circuits responsible for control, calculations, and autonomous decisions for this optical coherence tomography system. Light source 502 may be comprised of a broadband light source such as a superluminescent diode or tunable swept source laser system such as a vertical-cavity surface-emitting laser (VCSEL). The center wavelength for light source 502 can be suitable for optical coherence tomography of the eye, such as 840 nm, 1040 nm, 1050 nm, 1060 nm, 1310 nm or other wavelengths. The sweep range or spectral bandwidth of light source 502 may be as narrow as 20 nm, as wide as 120 nm, anywhere in the range between 20 nm and 120 nm or larger than 120 nm. Other sweep ranges are possible. In some embodiments, light source 502 can output light when sweeping in one direction, such as up or down, through its sweep range. In other embodiments, light source 502 can output light when sweeping in multiple directions, such as up and down, through its sweep range.

In some embodiments, light source 502 may be comprised of its own electronics which can be electronically controlled via an electrical connection to control electronics 500, such that light source 502 can be turned on, off or variably attenuated by electrical signals received through an electrical connection from control electronics 500. In other embodiments, the electronics in control electronics 500 may be responsible for electronic control of light source 502 through an electrical connection between control electronics 500 and light source 502. The electrical control signals from control electronics 500 to light source 502 may be single electrical signals or repeated signals at frequencies such as 1 Hz, 100 Hz, 1 kHz, 10 kHz, 50 kHz, 100 kHz, 200 kHz, 400 kHz, or 1 MHz. Other control frequencies and many voltages are possible. In embodiments where light source 502 is comprised of its own electronics, the electronics in light source 502 may generate single or repeated electrical signals at frequencies such as 1 Hz, 100 Hz, 1 kHz, 10 kHz, 50 kHz, 100 kHz, 200 kHz, 400 kHz, or 1 MHz. Other frequencies are possible. In various embodiments, these electrical signals from control electronics 500 or from light source 502 can also be called trigger signals and can indicate when light source 502 is beginning a sweep of its spectral range, such as rapid tuning of the wavelength of light output from light source 502 across its sweep range, such as from 1010 nm to 1110 nm. In some embodiments, these sweeps happen repeatedly with little time elapsing between successive wavelength sweeps. In other embodiments, the sweeps occur with intervals of other activity, such as sweeping in the opposite direction or non-sweeping activity, between them. In various embodiments, these electrical signals are output from light source 502 as trigger signals to synchronize with OCT data capture and other device operations.

Light from light source 502 may be coupled into a fiber optic, such as a single-mode or multi-mode fiber, or travel in free space to fiber coupler 504 such that the light from light source 502 may be split into two optical paths. As used herein, “coupler” may include splitters that split light into more than one light path as well as couplers which combine light from more than one light path into one or more light paths. The portion of light traveling through one output optical path from coupler 504 may contain a portion, such as 1%, 5% or 10%, of the input light from light source 502 while the other portion of light from light source 502, such as 99%, 95% or 90% respectively, may emanate through the other optical path output from coupler 504. The light traveling through one output optical path from coupler 504, such as the optical path with less light or the optical path containing more light, can travel through interferometer 506 which can be a Mach Zender interferometer or other type of interferometer. Light from interferometer 506 can be combined and sensed at detector 508, which may be a balanced detector or other type of detector that senses light and transforms the sensed light into an electrical signal using one or a plurality of photodetectors.

The electrical signal from detector 508 can be transmitted to electronics on control electronics 500 or other components in the system and used in the transformation and analysis of optical coherence tomography data. In many embodiments, the electrical signal from detector 508 can be used as a k-clock to indicate the spectral spacing of the wavelength sweep in light source 502. In some embodiments, the electrical signal from detector 508 can be modified, such as by ‘squaring’ the waveform, to transform it into a more usable k-clock signal. In other embodiments, this electrical signal can be sampled and captured on a data acquisition system, such as by electronics contained in control electronics 500. The data from this k-clock sampling can be analyzed using commonly-used signal analysis processes to determine the spectral spacing of the wavelength sweep occurring in light source 502. In still other embodiments, the k-spacing of the spectral sweep for light source 502 can be captured, modeled and subsequently artificially reproduced by electronics, such as a field programmable gate array (FPGA), on light source 502 or control electronics 500. And in some embodiments, the electrical signal from detector 508 can be transmitted to control electronics 500 and used in the laser power safety system to ensure that all lasers are operating within Class 1 limits.

The k-clock electrical signal from detector 508 can be captured all of the time, nearly all of the time or at discrete times after which it could be stored and recalled for use in OCT capture. In some embodiments, various parameters of the k-clock signal, such as frequency or voltage, can be modified electronically, such as doubled or quadrupled, to enable deeper imaging in eye tissues. In various embodiments with light sources that sweep in a substantially linear fashion, the k-clock can be removed and a linear sweep between trigger signals can be used in place of a sampled k-clock. In various embodiments, the trigger signals used by electronics 595r and 595l may be synchronized with other components of the system, such as mirrors, variable focus lenses, air pumps and valves, pressure sensors and flow sensors.

Beam coupler 510 can split the light from coupler 504 or light source 502 to two output optical paths, specifically right and left, that lead directly to couplers 515r and 515l. Henceforth, designation of a device or component with a suffix of ‘r” or ‘l’ will refer to two devices that may be of the same type but are located in different optical paths. For example, one component may be located in the optical path of the right eye, designated as ‘r,’ and the other is located in the optical path of the left eye, designated as ‘l.’ In some embodiments, light from coupler 504 may be attenuated, either with fixed or variable attenuation, to alter the amount of light being transmitted to coupler 510. In other embodiments, light from beam coupler 510 may be attenuated, either with fixed or variable attenuation, to alter the amount of light being transmitted to each of couplers 515r and 515l.

The optical paths in this system may be comprised of fiber optics, free space optics, or a mixture of free space and fiber optics. Other combinations are also possible. The split ratio of coupler 510 can be a predefined ratio, such as 50/50, 60/40 or 70/30. Light from coupler 510 can travel to couplers 515r and 515l. Couplers 515r and 515l may also split light from coupler 510 with a predefined split ratio such as a 50/50, 60/40, 70/30, or 90/10. The split ratios for couplers 510, 515r and 515l may be the same or different split ratios.

One portion of light from couplers 515r and 515l, such as 70%, can travel to a so-called ‘reference arm’ for each of the right and left optical paths. The reference arm of a light path is distinguished from the so-called sample arm of the light path since light in the reference arm of the system does not interface with eye tissue directly whereas light in the sample arm is intended to contact eye tissue directly.

The reference arm may be comprised of an optical delay device, labeled as 516r and 516l in the right and left optical paths of the system. Optical delay devices can introduce a time delay, such as 1 picosecond, 10 picoseconds or 100 picoseconds, into a light path to enable matching of the overall path length of one optical path to the optical path length of another light path. In various embodiments, this optical delay may be adjustable, such as with an adjustable free light path between two collimating optical devices, a fiber stretcher that increases or decreases the length of a fiber optic, or a fiber Bragg grating that delays light based on changes in the angle of incidence of light.

In other embodiments, this optical delay line can include variable attenuators to decrease or increase the transmission of light, optical switches or mechanical shutters to entirely block or unblock the light path. Although pictured in the reference arm of this system, an optical delay line can also be entirely included in the sample arm optical path for each eye or contained in both the reference and sample arm light paths. Other combinations of sample and reference light paths are also possible.

In one embodiment, light from optical delay devices 516r and 516l can travel to couplers 517r and 517l where it may be combined with light from the sample arm that has been transmitted from couplers 515r and 515l. Couplers 517r and 517l may combine light from two light paths with a predefined ratio between paths such as a 50/50, 70/30, or 90/10. Light from couplers 517r and 517l may travel through two outputs from couplers 517r and 517l to balanced detectors 518r and 518l where the light signal can be transformed into an electrical signal, for example through the use of photodiodes configured to detect the light input from couplers 517r and 517l.

The electrical signal generated by balanced detectors 518r and 518l can be in various ranges, including but not limited to −400 mV to +400 mV, −1V to +1V, −4V to +4V and have various bandwidths, including but not limited to 70 MHz, 250 MHz, 640 MHz, and 1.25 GHz. The electrical signal from balanced detectors 518r and 518l may travel via an electrical connection, such as a coaxial cable, to electronics 595r and 595l where it can be captured by a data acquisition system configured to capture data from balanced detector devices. In some embodiments, electronics 595r and 595l may be electrically coupled to control electronics 500 through flexible cables such as coaxial cables. In other embodiments, electronics 595r, 595l and 500 are all contained on the same printed circuit board and are electrically coupled via on-board circuitry.

Although not pictured here, a polarization sensitive optical component can be disposed before balanced detectors 518r and 518l to split two polarities of light in a single light path into two optical paths. In this embodiment, two optical paths leading to balanced detectors 517r and 517l would be split into a total of four optical paths which would lead to two balanced detectors on each side.

One portion of light from couplers 515r and 515l, such as 30%, 40% or 50%, can travel to a so-called sample arm of each of the right and left optical paths. In various embodiments, the system may be configured to transmit the light through fiber optic cable or through free space optics. If couplers 515r and 515l are fiber optic couplers, light from couplers 515r and 515l can travel to collimators 520r and 520l configured to collimate the light from output fiber optics emanating from couplers 515r and 515l. In some embodiments, collimators 520r and 520l have a fixed focal length. In other embodiments, the focal length of collimators 520r and 520l can be configured to vary, such as by mechanical motion of lenses in the collimators, while still producing a collimated beam of light in all or some of the variable focal length configurations. These embodiments have the advantage of enabling alterations in OCT focal depth and OCT lateral resolution. Examples of focal length ranges for these variable focal length collimators could include 3 mm to 18 mm, 6 mm to 12 mm, or 4 mm to 8 mm. Other focal length ranges are possible. In still other embodiments, the focal length of collimators 520r and 520l can be configured to vary, such as by mechanical motion of lenses in the collimators, without producing a collimated beam of light. In these embodiments, collimators 520r and 520l may produce a beam of light with non-zero vergence.

Light from optics 520r and 520l can travel to lens systems 525r and 525l which may be comprised of fixed focus or variable focus lenses. In various embodiments, these lenses can be fabricated from plastic or glass and modify the focus of the light path through mechanical motion. In other embodiments, these lenses may be electrowetting lenses or shape-changing lenses, such as fluid-filled lenses, that can vary their focal distance based on internal or external control mechanisms. In one embodiment, variable focus lenses in lens systems 525r or 525l may have their focal length modified by electrical current or voltage applied to lens systems 525r or 525l. This control may come from electrical components 595r and 595l and the parameters of this control may be based on pre-determined values or may be derived during operation of the system based on input received from other components of the system. The control signal to vary the focal length of lens systems 525r and 525l may be modified based on lens temperature, such as from temperature sensors embedded in the lenses, or physical properties of the lens such as its horizontal or vertical mounting orientation.

The lenses in lens systems 525r and 525l can be configured to have anti-reflective coatings, position feedback sensors, or other associated circuitry. Lens systems 525r and 525l may be comprised of a single lens or multiple lenses. One or more lens in lens systems 525r and 525l may move under the control of electronics 595r and 595l respectively in a linear or non-linear fashion with or without position feedback. The lenses comprising systems 525r and 525l may be present at all times or may be mechanically moved in and out of the light path such as by an attached motor and drive circuit under electrical control from electronics 595r and 595l. Configuration of lens systems 525r and 525l to be moveable can enable imaging at different depths in an eye tissue.

In some embodiments, light from lens systems 525r and 525l reflects directly off movable mirrors 530r and 530l. In other embodiments, light from lens systems 525r and 525l reflects off fixed mirrors 528l and 528r before reflecting off movable mirrors 530r and 530l. In these embodiments, the purpose of mirrors 528l and 528r is to reduce the angle of incidence of reflection of incident light on mirrors 530r and 530l. Movable mirrors 530r and 530l may be comprised of one or more MEMS (microelectromechanical systems) mirrors, galvanometer mirrors, or mirrors moved by other means. Movable mirrors 530r and 530l can be comprised of a single mirror that reflects light across 2 axes, such as X and Y, can be comprised of a single mirror that reflects light across one axis only, or can be comprised of two mirrors that each reflect light across one axis only said axes being substantially perpendicular to each other. In various embodiments, movable mirrors 530r and 530l may be enclosed by glass to exclude foreign contaminants such as dust. In other embodiments, all components 520l-530l are enclosed as a group by glass to exclude foreign contaminants and all components 520r-530r are enclosed as a group by glass to exclude foreign contaminants.

In some embodiments, at least one moving axis of movable mirrors 530r and 530l are electrically controlled by electronics 595r and 595l. The electronic control of mirrors 530r and 530l may be configured to enable variable amplitude deflections of mirrors 530r and 530l. For example, for a given drive frequency in a given axis, the current or voltage applied to mirrors 530r and 530l may enable larger or smaller amplitude deflections of the mirror surface, thus creating a zoom effect where the created image can be made smaller or larger. In some embodiments, the movement of mirrors 530r and 530l can be coordinated with the trigger signal from light source 502 or control electronics 500 to synchronize acquisition of image data.

Light that has been reflected from movable mirrors 530r and 530l travels to lens systems 535r and 535l. Lens systems 535r and 535l may be comprised of one or more groups of fixed or variable focus lenses. Any one of these lens groups can be located in the optical light path at all times or only part of the time. In some embodiments, the position, location or state of lenses or lens groups in lens systems 535r and 535l is controlled by electronics 595r and 595l such as by moving one or more lens groups into or out of the light path or changing the focal length of one or more lenses in lens systems 535r and 535l. Other actions are also possible. In various embodiments, the lens barrels within lens systems 535r and 535l can be configured with shields that extend beyond the outer diameter of the lens barrel, said shields configured to cover the space between lens systems 535r and 540r or 535l and 540l to reduce entrance of foreign contaminants between these lens systems.

In some embodiments, lens systems 535r and 535l may also be comprised of optical systems that do not transmit light from movable mirrors 530l and 530r and are comprised of their own light sources and sensing optics, such as for determining the wavefront aberrations or refractive error of the eye in the sample arm optical path. The light sources in these optics can be of a suitable wavelength to interact with optics 540r and 540l in the same way or a substantially similar way as other light transmitted by lens systems 535r and 535l. The light source and sensor in these alternative optical systems can be electrically coupled, such as for electrical control or data transfer, to electronics 595r and 595l. These alternative optics in lens systems 535r and 535l can be comprised of beamsplitters, collimating optics, focusing optics, or multi-lenslet arrays.

Light from lens systems 535r and 535l can travel to optics 540r and 540l which may be comprised of dichroic mirrors or couplers. In some embodiments, optics 540r and 540l may be configured to transmit light from lens systems 535r and 535l and combine it with light reflected from lens systems 545r and 545l. In other embodiments, optics 540r and 540l may be configured to reflect light from lens systems 535r and 535l and combine it with light transmitted from lens systems 545r and 545l. Optics 540r and 540l may be configured with coatings suitable to transmit and reflect specific wavelengths of light efficiently. Optics 540r and 540l can be configured to interact with light of widely varying wavelengths such as a range from 400 nm to 1200 nm. Optics 540r and 540l can also be configured to enable wide fields of light to transmit or reflect from it. If configured as a dichroic mirror, optics 540r and 540l can be configured with an angled edge that is substantially parallel to the direction of light being conveyed to optics 542r and 542l to maximize the clear aperture along the optical axis of optics 542r and 542l. In some embodiments, optical windows without any optical power can be configured adjacent to optics 540r and 540l to prevent foreign contaminants from settling onto optics 540r and 540l. In various embodiments, sensors 538r and 538l may be configured to receive light reflected off or transmitted through optics 540r and 540l, transform that light into an electrical signal and transmit that electrical signal through an electrical connection to electronics 595r and 595l or control electronics 500.

Light from optics 540r and 540l travels through eye pieces 542r and 542l before being transmitted to the right and left eye tissues. Eye pieces 542r and 542l can be configured as multi-element lens systems such as Ploessel-type eyepieces, Erfle-type eyepieces, telescopes or other designs. In some embodiments, optics 540r and 540l may be configured to be part of or inside of eyepieces 542r and 542l. In other embodiments, variable focus lenses or polarization-sensitive optics and beam splitters can be configured inside eyepieces 542r and 542l to enable wider axial focusing ranges in eye tissues or simultaneous focusing of light from two axial locations in eye tissues. Eyepieces 542r and 542l may be configured with optical components without any refractive power, such as optical windows, that may be physically attached or separate from the other lenses in the system.

In many embodiments, optics 544r and 544l are configured to be placed between optics 542r and 542l and the eye. Optics 544r and 544l prevent foreign contaminants from the eye from reaching optics 542r and 542l. The curved surface of optics 544r and 544l reduces backreflections from propagating to balanced detectors 518r and 518l and sensors 562r-565r and 565l-562l. In various embodiments, optics 544r and 544l are comprised of thin, disposable plastic such that they can be single-use. Optics 544r and 544l are configured to have no optical power. Optics 544r and 544l can be configured to introduce minimal aberrations, such as less than 0.07 rms waves, into the optical path. Optics 544r and 544r can also be configured to be as thin as possible, such as 0.3 mm, 0.5 mm or 0.7 mm, without becoming distorted. Various methods of reversibly attaching optics 544r and 544l to the system are discussed in other applications.

Light entering the right and left eyes can be reflected back through each optical path to enable optical coherence tomography. In one embodiment, the path of backreflected light originating from light source 502 can travel from each eye through optics 544 to eyepiece 542, to optics 540, to lens system 535, to movable mirror 530, to fixed mirror 528, to lens system 525, to optics 520, to coupler 515, to coupler 517 and to balanced detector 518. Various calculations and logic-based processes can be completed by components 595r and 595l based on data contained in signals received from balanced detectors 518r and 518l.

As discussed previously, timing of capture of the signals received by components 595r and 595l may be controlled by other inputs, such as the k-clock input, a dummy clock input, or other electrical signal such as the trigger signal. Electronics 500, 595r, and 595l may be configured to have digital signal processors (DSPs), field-programmable gate arrays (FPGAs), ASICs or other electronics to enable faster, more efficient or substantially real-time processing of signals received by components 595r and 595l. Electronics 500, 595r, and 595l may be configured with software, such as a real-time operating system, to enable rapid decisions to be made by said components. Electronics 500, 595r and 595l can also be configured to sense the power of light being projected onto the eye and to reduce the power output from light source 502 if safety limits are exceeded.

In various embodiments not illustrated here, the eye tissues may be replaced by calibration targets that, for example, occlude the eyepieces, dispose a mirror target at various distances in front of optics 542, dispose a diffusely reflective target at various distances in front of optics 542, dispose a structured target, such as a grid or set of lines, at various distances in front of optics 542, or provide an open air space for calibration. Electronics 500 may be configured to control the introduction of these non-tissue targets, such as when the eyes are not present in the optical system. In other embodiments, electronics 500 may be configured to dispose moveable components of the system to various states, such as “off,” “home,” or “safety” at various times, such as the beginning, middle and end of a test.

Components 595r and 595l can also be configured to control light sources 596r-599r and 596l-599l which may be comprised of laser diodes, light emitting diodes, or superluminescent diodes of different wavelengths and powers. In the illustrated embodiment, only four light sources 596r-599r and 596l-599l are shown on each side. In one embodiment, light source 596 is centered at or near 450 nm, light source 597 is centered at or near 520 nm, light source 598 is centered at or near 635 nm, and light source 599 is centered at or near 780 nm. Other center wavelengths are possible for these light sources. In other embodiments, fewer than four or more than four light sources may be used.

In some embodiments, light sources 596r-599r and 596l-599l may be comprised of tunable light sources capable of producing numerous spectra of light from a single source. In these embodiments, fewer than four light sources may be required for each eye. In addition to functioning as a display source, the light sources in these embodiments may be used for the purpose of hyperspectral imaging. For example, employing various light sources in the visible spectrum capable of producing narrow bands of light centered at characteristic peaks of absorption or reflectivity for oxyhemoglobin and deoxyhemoglobin can be used to enable hyperspectral imaging. Similarly, numerous individual discrete light sources can be used to achieve the same effect as a light source with a tunable wavelength.

Light sources 596r-599r and 596l-599l can be configured to be controlled by electronics 595r and 595l using, for example, pulse-width modulation, current modulation, voltage modulation, or other electrical control means. The modulation rate of said control by electronics 595r and 595l may be in the range of 1 MHz to 120 MHz, 2 Mhz to 240 Mhz, or 100 MHz to 140 MHz. Other modulation rates are possible. In one embodiment, the modulation frequency of at least one light source can be modified to correct for chromatic aberration from the optics between the light sources and the eye. For example, the modulation frequency of the red channel could be variably increased or decreased in different mirror positions to account for lateral chromatic spread between the red light source and other colors such as blue or green.

In various embodiments, light sources 596r-599r and 596l-599l are laser diodes coupled to single-mode fibers, such as with micro-optics, for the purpose of producing a near-Gaussian wavefront in the single-mode fibers. In other embodiments, light sources 596r-599r and 596l-599l are laser diodes that emit light into free space for shaping with optics such as anamorphic prism pairs and focusing optics. In some embodiments, light sources 596r-599r and 596l-599l are coupled to temperature-stabilizing electromechanics capable of both sensing and manipulating the thermal environment of the light source, such as with a Peltier device. In other embodiments, light sources 596r-599r and 596l-599l are coupled to temperature sensing electronics that provide electrical signals to electronics 595r and 595l to modify the drive signal of light sources 596r-599r and 596l-599l based on their measured temperature.

Light from light sources 596r-599r and 596l-599l can be coupled together by optics 590r and 590l which may in some embodiments be free space couplers and in other embodiments be fiber-based couplers. Light from optics 590r and 590l travels to collimating optics 585r and 585l capable of transmitting wavelengths in the range of 400 nm to 800 nm. Optics 580r and 580l accept light from optics 585r and 585l and may be dichroic mirrors or couplers configured to have a pre-defined split ratio of light entering from different directions or having different wavelengths, such as 90/10, 50/50, and 10/90. A portion of the light reflected from optics 580r and 580l may be received at photodetector 582r and 582l which may be coupled electronically to electronics 595r and 595l which can process the signal received from photodetectors 582r and 582l before passing those data along to control electronics 500 for further processing.

The portion of the light path that passes through optics 580r and 580l is received by optics 575r and 575l which, in some embodiments, are capable of introducing cylinder power at an arbitrary axis into the light path. Optics 575r and 575l can be comprised of paired cylindrical lenses configured to counter-rotate and co-rotate with each other, an electrically controlled variable focus, liquid filled lens, or other method of introducing cylindrical optical power into a light path. In various embodiments, optics 565r and 565l can be configured as dichroic mirrors or couplers configured to have a pre-defined split ratio of light entering from different directions or having different wavelengths, such as 90/10, 50/50, and 10/90.

A portion of light from optics 565r and 5651 can be transmitted through to optics 555r and 5551 which are optional color correction optics. Fold mirrors 554r and 5541 are inserted into the light path to make the system smaller in size and direct light onto optics 553r and 5531 which may be comprised of fixed focus or variable focus lenses. In various embodiments, optics 553r and 5531 can be fabricated from plastic or glass and modify the focus of the light path through mechanical motion. In other embodiments, these lenses may be electrowetting lenses or shape-changing lenses, such as fluid-filled lenses, that can vary their focal distance based on internal or external control mechanisms. In one embodiment, variable focus lenses in lens systems 553r or 5531 may have their focal length modified by electrical current or voltage applied to lens systems 553r or 553l. This control may come from electrical components 595r and 595l and the parameters of this control may be based on pre-determined values or may be derived during operation of the system based on input received from other components of the system. The control signal to vary the focal length of lens systems 553r and 5531 may be modified based on lens temperature, such as from temperature sensors embedded in the lenses, or physical properties of the lens such as its horizontal or vertical mounting orientation.

In some embodiments, light from lens systems 553r and 5531 reflects off fixed mirrors 5521 and 552r before reflecting off movable mirrors 550r and 550l. In these embodiments, the purpose of mirrors 5521 and 552r is to reduce the angle of incidence of reflection of incident light on mirrors 550r and 550l. In other embodiments, apertures stops are inserted into the light path between optics 553r and 5531 and mirrors 552r and 552l. Movable mirrors 550r and 5501 may be comprised of one or more MEMS (microelectromechanical systems) mirrors, galvanometer mirrors, or mirrors moved by other means. Movable mirrors 550r and 5501 can be comprised of a single mirror that reflects light across 2 axes, such as X and Y, can be comprised of a single mirror that reflects light across one axis only, or can be comprised of two mirrors that each reflect light across one axis only said axes being substantially perpendicular to each other. In various embodiments, movable mirrors 550r and 5501 may be enclosed by glass to exclude foreign contaminants such as dust. In other embodiments, all components 553l-550l are enclosed as a group by glass to exclude foreign contaminants and all components 553r-550r are enclosed as a group by glass to exclude foreign contaminants.

In some embodiments, at least one moving axis of movable mirrors 550r and 5501 are electrically controlled by electronics 595r and 595l. Movable mirrors 550r and 5501 may be configured with one axis of fast resonant movement, one axis of slow resonant movement, two slow axes of movement, one fast resonant axis and one slow axis of movement, or two fast resonant axes of movement. The electronic control of mirrors 550r and 5501 may be configured to enable variable amplitude deflections of mirrors 550r and 550l. For example, for a given drive frequency in a given axis, the current or voltage applied to mirrors 550r and 5501 may enable larger or smaller amplitude deflections of the mirror surface, thus creating a zoom effect where the created image can be made smaller or larger. In some embodiments, the movement of mirrors 550r and 550l can be coordinated with the trigger signal from light sources 596r-599r and 596l-599l or control electronics 500 to synchronize both drawing and capture of reflected light.

Light that has been reflected from movable mirrors 550r and 5501 travels to lens systems 545r and 545l. Lens systems 545r and 5451 may be comprised of one or more groups of fixed or variable focus lenses. Any one of these lens groups can be located in the optical light path at all times or only part of the time. In some embodiments, the position, location or state of lenses or lens groups in lens systems 545r and 5451 is controlled by electronics 595r and 595l such as by moving one or more lens groups into or out of the light path or changing the focal length of one or more lenses in lens systems 545r and 545l. Other actions are also possible. In various embodiments, the lens barrels within lens systems 545r and 5451 can be configured with shields that extend beyond the outer diameter of the lens barrel, said shields configured to cover the space between lens systems 545r and 540r or 5451 and 540l to reduce entrance of foreign contaminants between these lens systems.

Light from lens systems 545r and 5451 can travel to optics 540r and 540l which may be comprised of dichroic mirrors or couplers. In some embodiments, optics 540r and 540l may be configured to reflect light from lens systems 545r and 5451 and combine it with light reflected from lens systems 535r and 535l. In other embodiments, optics 540r and 540l may be configured to transmit light from lens systems 545r and 5451 and combine it with light transmitted from lens systems 535r and 535l. Optics 540r and 540l may be configured with coatings suitable to transmit and reflect specific wavelengths of light efficiently. Optics 540r and 540l can be configured to interact with light of widely varying wavelengths such as a range from 400 nm to 1200 nm. Optics 540r and 540l can also be configured to enable wide fields of light to transmit or reflect from it. In some embodiments, optical windows without any optical power can be configured adjacent to optics 540r and 540l to prevent foreign contaminants from settling onto optics 540r and 540l.

As discussed previously, light from optics 540r and 540l travels through eye pieces 542r and 5421 and optics 544r and 544l before being transmitted to the right and left eye tissues where it can be configured to be seen by the subject as organized light, such as in a retinal scanning display system. Modulation of light sources 596r-599r and 596l-599l can be configured to enable video displays. Light entering the right and left eyes can also be reflected back through each optical path to enable scanning laser ophthalmoscopy imaging. In one embodiment, the path of backreflected light originating from light sources 596r-599r and 596l-599l can travel from each eye through optics 544 to eyepiece 542, to optics 540, to lens system 545, to movable mirror 550, to fixed mirror 552, to lens system 553, to fixed mirror 554, to optics 555, to dichroic mirror 565 where portions are reflected to optics 564r and 564l which may be configured as focusing optics. Light from focusing optics 564r and 564l can be configured to reflect off of fixed aiming mirrors 563r and 5631 before being projected through a pinhole disposed between mirrors 563r and 5631 and photodiodes 561r and 561l. These photodiodes, which may also be comprised of avalanche photodiodes, photon multiplier tubes, or other highly amplified photodetectors, are electrically coupled to electronics 595r and 595l which are comprised of electronics capable of capturing high speed, such as 10 MHz, 100 MHz or 220 MHz, data from photodetectors 561r and 561l.

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or logic circuitry that implements a state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements or steps. Thus, such conditional language is not generally intended to imply that features, elements or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A system configured to de-identify healthcare data from an ophthalmic diagnostic device, the system comprising:
a computing system in electronic communication with the ophthalmic diagnostic device, the computing system comprising a processor and computer-executable instructions, wherein the computer-executable instructions, when executed, cause the computing system to:
process individually-identifiable eye examination data received from the ophthalmic diagnostic device;
request and receive at least one ordered value from a remote location in electronic communication with said computing system; the at least one ordered value being based at least in part on the order generated and not based on the specific date or time of the request or the individually-identifiable eye examination data, the at least one ordered value being larger than previously generated ordered values from the remote location, and the at least one ordered value being smaller than future generated ordered values from the remote location;
replace at least one date or portion of a date in the individually-identifiable eye examination data with the at least one ordered value;
and output de-identified healthcare data incorporating the at least one ordered value.

2. The system of claim 1, wherein the at least one ordered value is numeric.

3. The system of claim 1, wherein the at least one ordered value is non-numeric.

4. The system of claim 1, wherein the at least one ordered value is received less than one second from the request.

5. The system of claim 1, further comprising secure communication between the system and the remote location providing the at least one ordered value.

6. The system of claim 1, wherein each of the at least one ordered value is offset by an arbitrary increment.

7. The system of claim 1, wherein the de-identified healthcare data output can be viewed on a computer.

8. The system of claim 1, wherein the de-identified healthcare data output can be viewed with reference intervals demonstrating the limits of normal values.

9. A system configured to de-identify healthcare data from an ophthalmic diagnostic device, the system comprising:
a computing system in electronic communication with the ophthalmic diagnostic device, the computing system comprising a processor and computer-executable instructions, wherein the computer-executable instructions, when executed, cause the computing system to:
process individually-identifiable eye examination data received from the ophthalmic diagnostic device;
request and receive at least one ordered value from a remote location in electronic communication with said computing system; the at least one ordered value being based at least in part on the order generated and not based on the specific date or time of the request or the individually-identifiable eye examination data, the at least one ordered value being larger than ordered values generated from the remote location since the beginning of the calendar year, and the at least one ordered value being smaller than ordered values generated from the remote location for the remainder of the calendar year;
replace at least one date or portion of a date in the individually-identifiable eye examination data with the at least one ordered value;
and output de-identified healthcare data incorporating the at least one ordered value.

10. The system of claim 9, wherein the at least one ordered value is numeric.

11. The system of claim 9, wherein the at least one ordered value is non-numeric.

12. The system of claim 9, wherein the at least one ordered value is received less than one second from the request.

13. The system of claim 9, further comprising secure communication between the system and the remote location providing the at least one ordered value.

14. The system of claim 9, wherein each of the at least one ordered value is offset by an arbitrary increment.

15. The system of claim 9, wherein the de-identified healthcare data output can be viewed on a computer.

16. The system of claim 9, wherein the de-identified healthcare data output can be viewed with reference intervals demonstrating the limits of normal values.

17. A system configured to assist in the de-identification of healthcare data comprising:
a computing system comprising a processor and computer-executable instructions, wherein the computer-executable instructions, when executed, cause the computing system to:
process a request across from a remote location to generate at least one ordered value;
generate the at least one ordered value such that the value is larger than any previous value generated by said system, is smaller than any future value to be generated by said system and the value is unrelated to the time of the request or calculation;
output said ordered value to the remote location in less than one second from receiving the request.

18. The system of claim 17, wherein the at least one ordered value is numeric.

19. The system of claim 17, wherein the at least one ordered value is non-numeric.

20. The system of claim 17, further comprising secure communication between the system and the remote location.

21. The system of claim 17, wherein each of the at least one ordered value is offset by an arbitrary increment.
